(19) United States
(12) Patent Application Publication
(10) Pub. No.: US 2013/0243846 A1
(43) Pub. Date: Sep. 19, 2013

(54) PRESS-CONTACT TYPE IMMERSING INTERLAYER TISSUE

(71) Applicant: Dezheng Zhao, Wulumuqi (CN)

(72) Inventor: Dezheng Zhao, Wulumuqi (CN)

(21) Appl. No.: 13/811,266

(22) PCT Filed: Apr. 19, 2011
(86) PCT No.: PCT/CN11/73035

§ 371 Date: Jun. 3, 2013

(30) Foreign Application Priority Data
Jul. 20, 2010 (CN) 201020263425.9

(51) Int. Cl. A47K 7/03; A61K 9/70

(52) U.S. Cl. CPC A47K 7/03; A61K 9/70
USPC 424/443; 428/313.3; 442/393; 428/76; 428/158

(57) ABSTRACT

A press-contact type soaked interlayer tissue includes face towel sheets. The face towel sheet comprises two layers of face towel sheet parts (4), (6) and hollow balls (2),wherein the shell wall of the hollow ball is made of organic degradable plastics film The shell wall of the hollow ball (2) is in fully enclosed condition and the hollow ball has spherical or elliptic shape. The respective surrounding inner wall surfaces of the two layers of face towel sheet parts (4), (6) are connected with each other by adhering. The two layers of face towel sheet parts (4), (6) are made of tissue-paper or non-woven fabrics. The hollow balls (2) are arranged between the two layers of face towel sheet parts (4), (6) in matched manner. Liquid (5) nontoxic to human skin is sealed in the hollow balls (2).

### TECHNICAL FIELD

This utility model belongs to an improvement of the ordinary wet tissue structure, especially a press-contact type immersing interlayer tissue.

### TECHNICAL BACKGROUND

Presently, ordinary wet tissue needs to be individually packaged in a sealed plastic pouch after soaking. Otherwise, it will not be able to remain in a moist state. When used, an adhesive strip on the seal pouch needs to be lifted so that the package can be opened to remove the wet tissue before it can be used to wipe body parts, such as hands and arms, or other objects. If there is a crack or an opening on the sealed package, liquid contained in the wet tissue can easily volatize or evaporate from the crack or opening. Once a wet tissue is removed from its package, if it is not used in a timely manner, liquid contained in the wet tissue will also vaporize or evaporate within a short period of time. As a result, it is more difficult and less convenient to use. In addition, wet tissue cannot be utilized effectively or repeatedly. Due to the fact that it is inconvenient to use, its application is limited to a single use as a disposable product only.

### CONTENT OF UTILITY MODEL

The object of this utility model is to provide a press-contact type of immersing interlayer tissue. It is reasonably structured and is more convenient to use. There is no need to keep it in a sealed pouch to keep it in a moist state. As a result, there is a wide range of applications.

The object of this utility model is achieved as follows: A press-contact type of immersing interlayer tissue, including a facial tissue, is comprised of two facial tissue layers and hollow balls constructed with walls made of an organic degradable plastic film. The shell of the hollow ball is fully enclosed. It is either spherical or elliptical in shape. The inner peripheral walls of the two facial tissue layers are adhered to each other. The layers can either be made of tissue paper or non-woven cloth. Hollow balls are uniformly distributed in between these two tissue layers. A liquid that is not toxic to the human body is used to fill the inside of these hollow balls.

The key technology of this utility model involves the use of hollow balls in between the two layers of tissue paper. These hollow balls can be crushed by means of squeezing. The shell of the hollow ball is made of an organic degradable plastic film. A liquid that is not toxic to the human body, such as a perfume, disinfectant or hand sanitizer, may be contained inside these hollow balls. During the course of its use, some or all those hollow balls in between the two tissue paper layers are squeezed and crushed by hand. Liquid inside those crushed hollow balls will be discharged and will soak the facial tissue so that it can be used to wipe certain body parts, such as the skin, or other objects. The structure of this utility model is reasonable and it is more convenient to use. There is no need to use a sealed pouch to keep the facial tissue moist. It has a wide range of applications and it is highly adaptable to different situations.

### DESCRIPTION OF ATTACHED DRAWINGS

This utility model is explained in more detail in conjunction with the attached drawings as follows:

FIG. 1 is a schematic diagram of an external perspective view of the structure described in preferred embodiment 1 of this utility model.

FIG. 2 is a schematic diagram in section of the structure shown in FIG. 1.

FIG. 3 is a schematic diagram in section of the structure described in preferred embodiment 2 of this utility model.

FIG. 4 is a schematic diagram of an external perspective view of the structure described in preferred embodiment 3 of this utility model.

FIG. 5 is a schematic diagram of an external perspective view of the structure described in preferred embodiment 4 of this utility model.

FIG. 6 is a schematic diagram in section of the structure shown in FIG. 5.

FIG. 7 is a schematic diagram of a magnified sectional view of the semi-spherical shell described in preferred embodiment 4 of this utility model.

### DESCRIPTION OF PREFERRED EMBODIMENTS

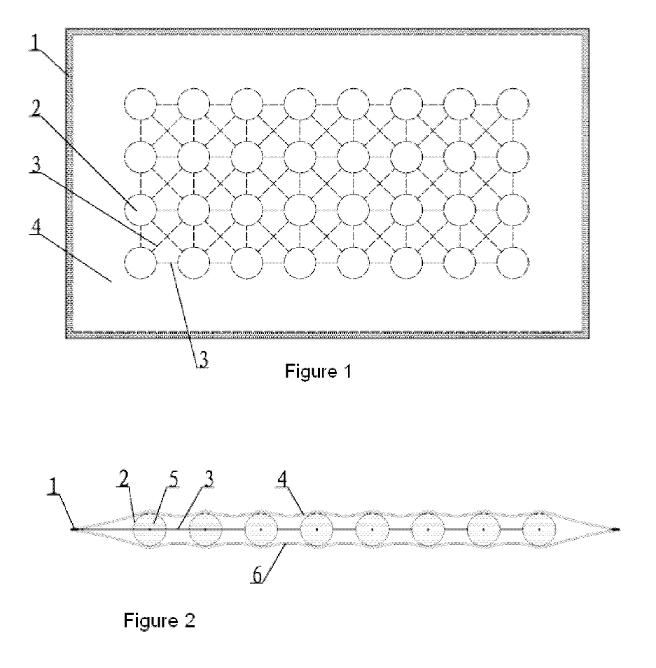

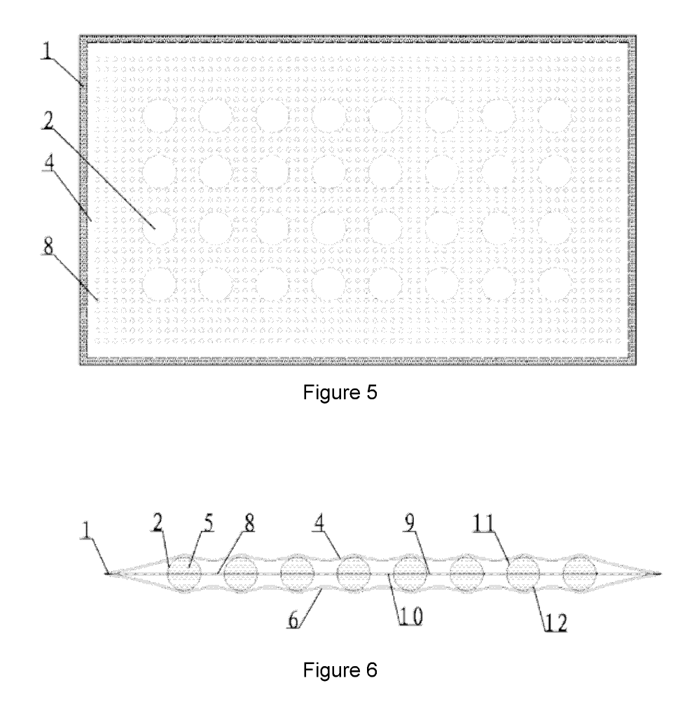

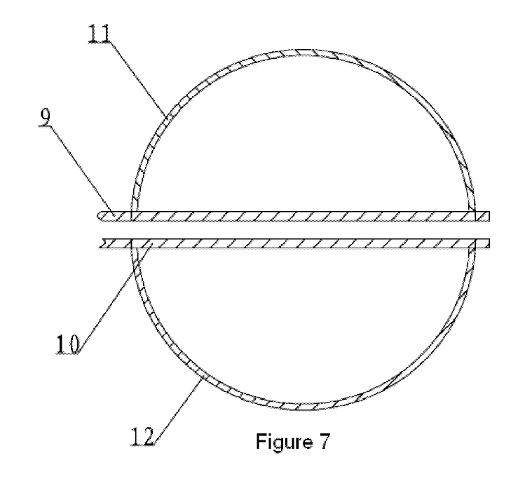

A press-contact type soaked interlayer tissue, as shown in FIGS. 1 through 7, comprises two layers of facial tissue (4) and (6), and hollow balls (2), wherein the shell wall of each hollow ball is made of an organic degradable plastic film. The shell wall of hollow ball (2) is in a fully enclosed condition and said hollow ball is either spherical or elliptical in shape. The respective peripheral inner wall surfaces of said two facial layers (4) and (6) are adhered to each other. Said two facial tissue layers (4) and (6) are either made of tissue paper or non-woven fabric. Said hollow balls (2) are arranged in between the two layers of facial tissue (4) and (6) in a matched manner. A liquid (5), which is nontoxic to human skin, is sealed inside said hollow balls (2).

Liquid (5) may be purified water, perfume, disinfectant, hand sanitizer, washing liquid or alcohol. A highly volatile liquid, such as alcohol, can be placed inside said hollow balls (2). For example, after said hollow balls (2) are crushed in an athletic field or on a hot day, the tissue can be used to wipe off the human skin as a single-use disposable product. Volatile liquid released by crushing said hollow balls (2) can produce a temperature-lowering and cooling effect. If a semi-permeable membrane is used to fabricate said hollow balls (2), and perfume is used to fill the inside of said hollow balls (2), its evaporation can be slowed down. Or, the tissue can be placed in upscale clothing to add a fragrance to the merchandise. Furthermore, a medicinal (medical) solution may be used to fill said hollow balls (2). After said hollow balls (2) are crushed, the medicinal (medical) solution can be smeared or wiped onto a wound or a swollen and painful spot to facilitate disinfection and sterilization.

Each spherically shaped hollow ball (2) can have a diameter of the order of 2-8 mm. Each elliptically shaped hollow ball (2) can have its longest diameter of the order of 3-9 mm.

As shown in FIGS. 1 through 4, each hollow ball (2) is connected to its nearest adjacent hollow balls (2) through the use of filaments (3).

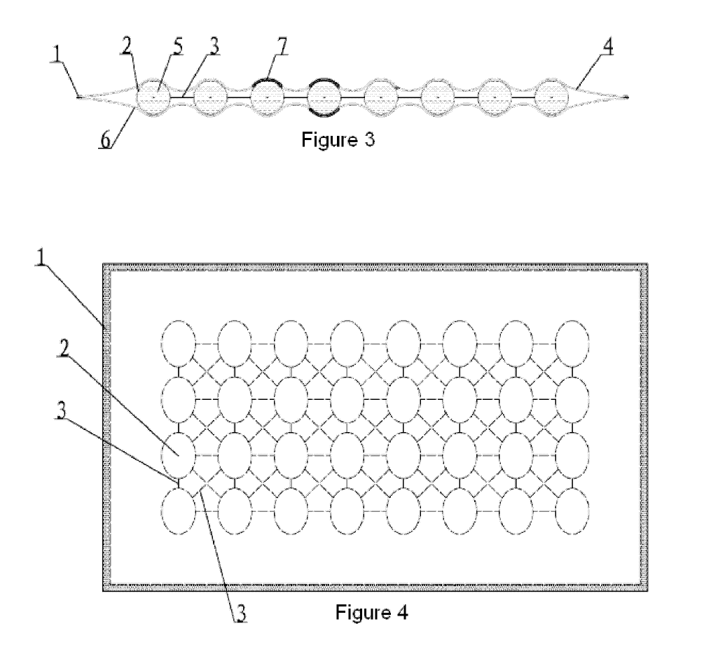

As shown in FIG. 3, spherical shells (7) are uniformly distributed on the two facial tissue layers (4) and (6). The surface of spherical shell (7) bulges outward from the external surface of the two facial tissue layers (4) and (6). The spherical shell on the facial tissue and its internal spherically concave surface should be matched with the external surface of each hollow ball (2) so that each hollow ball (2) can be fixed in a specific location between said two facial tissue layers.

As shown in FIGS. 5, 6 and 7, a double-layer overlapping film made of degradable plastic films (9) and (10) is placed in between two facial tissue layers (4) and (6). The periphery surrounding this double-layer overlapping film is sandwiched in between the inner peripheral walls of said two facial tissue layers (4) and (6). In addition, both sides of the wall around the periphery of the double-layer overlapping membrane are adhered to their corresponding inner walls. A degradable film is placed on the exterior surface of the bulging hemispherical shell. Degradable films (9) and (10) are bonded to form a double-layer overlapping film on the interior flat wall. Therefore, degradable films (9) and (10) can be used to form a double-layer overlapping film covering the hollow balls that are bulging on either side from two hemispherical shells (11) and (12) configured on two facial tissue layers. Each hollow ball (2) formed in between two facial tissue layers can then be fixed in place. A hole (8) is set up on the film so that both sides of the two-layer facial tissue can be effectively soaked.

As shown in FIGS. 1 through 5, a bonding surface (1) is set up on the inner peripheral wall surrounding the two facial tissue layers (4) and (6). This bonding surface (1) is used to bond the inner peripheral wall of tissue layer (4) to the inner peripheral wall of tissue layer (6).

1. A press-contact type of immersing interlayer tissue, including facial tissue, which is characterized by using two layers of facial tissue and hollow balls made of an organic degradable plastic thin film shell in its construction; the shell of said hollow ball is completely enclosed and it is either spherical or elliptical in shape; peripheral inner walls of said two facial tissue layers are adhered to each other and said tissue layers are made of tissue paper or non-woven fabric; said hollow balls are evenly distributed between said two tissue layers; and said hollow balls are filled with a liquid that is nontoxic to the human skin.

2. The press-contact type of immersing interlayer tissue as described in claim 1, further characterized by the fact that said liquid is purified water, perfume, disinfectant, hand sanitizer, washing liquid or alcohol.

3. The press-contact type of immersing interlayer tissue as described in claim 1, further characterized by the fact that each hollow ball may be spherical in shape and has a diameter of 2-8 mm, or it may be elliptically shaped with a longest diameter of 3-9 mm.

4. The press-contact type of immersing interlayer tissue as described in claim 1, further characterized by that fact that each hollow ball is connected to the nearest adjacent hollow balls through the use of a filaments.

5. The press-contact type of immersing interlayer tissue as described in claim 1, further characterized by the fact that spherical shells are uniformly distributed across said two tissue layers, said spherical shells bulging on the surface of said two tissue layers; the inner concave surface of each spherical shell on said tissue surface matches the outer surface of each hollow ball to fix in position each hollow ball placed in between said two tissue layers.

6. The press-contact type of immersing interlayer tissue as described in claim 1, further characterized by the presence of a double-layer overlapping film made of a pair of degradable plastics in between the two layers of facial tissue and, the periphery of said double-layer overlapping film being sandwiched in between the inner peripheral walls of said two facial tissue layers; moreover, both side walls of said double-layer overlapping film are adhered to the inner peripheral walls of said two facial tissue layers, respectively; there are bulging hemispheric shells on the outer surface of each degradable film surface; degradable plastic films are bonded to their respective inner flat wall to form said double-layer overlapping film so that hemispheric shells are formed on their corresponding sheets made of said degradable plastic films and these shells can be paired to form hollow balls made of said double-layer overlapping film and said hollow balls bulge out on both sides; each hollow ball formed in between two facial tissue layers is fixed in place, and holes are made on the double-layer overlapping film.

7. The press-contact type of immersing interlayer tissue as described in claim 1, further characterized by the presence of a bonding surface around the inner peripheral wall of said facial tissue layers, and the inner walls of said two facial tissue layers are adhered to each other via said bonding surface.
